(19) United States
(12) Patent Application Publication
(10) Pub. No.: US 2009/0037119 A1
(43) Pub. Date: Feb. 5, 2009

(54) System for estimation and control of the concentrations of pollutant gases at the discharge of a gas turbine

(76) Inventors: Gianni Mochi, Firenze (IT); Laura Cassi, Roma (IT); Luciano Bonciani, Firenze (IT); Gianni Ceccherini, Sesto Fiorentino (IT); Leonardo Rosi, Egidio (IT)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 10/540,374

(22) PCT Filed: Dec. 18, 2003
(86) PCT No.: PCT/EP03/14559

§ 371 Date: Nov. 30, 2007

(30) Foreign Application Priority Data
Dec. 23, 2002 (IT) MI2002A002752

(51) Int. Cl. G01N 33/00

(52) U.S. Cl. 702/24

(57) ABSTRACT

System for estimation and control of the concentrations of pollutant gases at the discharge of a gas turbine comprising a unit (3) for acquisition of the data relating to the operating state of the turbine, the said data being detected by a control panel (2) of the turbine. The system also comprises a local processing unit (4), which processes the said data in association with the said acquisition unit and makes the data available for consultation by the latter.

The present invention relates to a method and a system for estimation and control of the concentrations of pollutant gases at the discharge of a gas turbine.

In particular, the present invention relates to a method and a system for monitoring the emissions at the discharge of a gas turbine, without using continuous analysers of these emissions.

As is known, gas turbines are machines which consist of a compressor and a turbine with one or more stages, wherein these components are connected to one another by a rotary shaft, and wherein a combustion chamber is provided between the compressor and the turbine.

Air is supplied from the external environment to the compressor, in order to pressurise the latter.

By means of corresponding pipes, the pressurised air reaches the combustion chamber where, by means of one or more injectors which are supplied by a pressurised network, there is introduction of the fuel necessary to produce the combustion, which is designed to give rise to an increase in the temperature and enthalpy of the gas.

Via corresponding pipes, the high-temperature, high-pressure gas reaches the various stages of the turbine, which transforms the enthalpy of the gas into mechanical energy available to a user.

The monitoring systems of a known type control the emissions of the gas burnt in the turbine, by means of in-line analysers.

The applicant has observed that these analysers of the gas emissions require frequent calibration interventions, for example to be carried out by means of sample cylinders. In general, in each case it is necessary to carry out a periodic check of the compatibility of the results of the analyser, with the emissions actually measured, in order to keep the model compatible with the most recent states of the machine (long-term deterioration).

The applicant has created a system for estimation and control of the concentrations of pollutant gases at the discharge of a gas turbine, wherein, by means of analysis of a control panel of the turbine and calculation of the values detected by the panel, it is possible to deduce the emissions of the turbine (for example oxygen, nitric oxides, carbon monoxide). Thus, the system created does not need an in-line analyser for the emissions, which are predicted by means of analysis of the conventional values which can be detected by an operating panel of the machine.

One aspect of the present invention relates to a method for estimation and control of the concentrations of pollutant gases at the discharge of a gas turbine, comprising the following steps:
receipt of a plurality of signals corresponding to data relating to the operating state of the turbine;
processing of this data; and
evaluation of the emissions into the atmosphere from this turbine on the basis of the said data processed.

A further aspect of the present invention relates to a system for estimation and control of the concentrations of pollutant gases at the discharge of a gas turbine, characterised in that it comprises:
an acquisition unit for the data relating to the operating state of the turbine, the said data being detected by a control panel of the turbine; and
a local processing unit which processes the said data in association with the said acquisition unit and makes the data available for consultation, in order to evaluate the emissions by the said turbine into the atmosphere, on the basis of the said data processed.

The characteristics and advantages of the method and the system according to the present invention will become clearer and more apparent from the following description, provided by way of non-limiting example, with reference to the attached figures, in which:

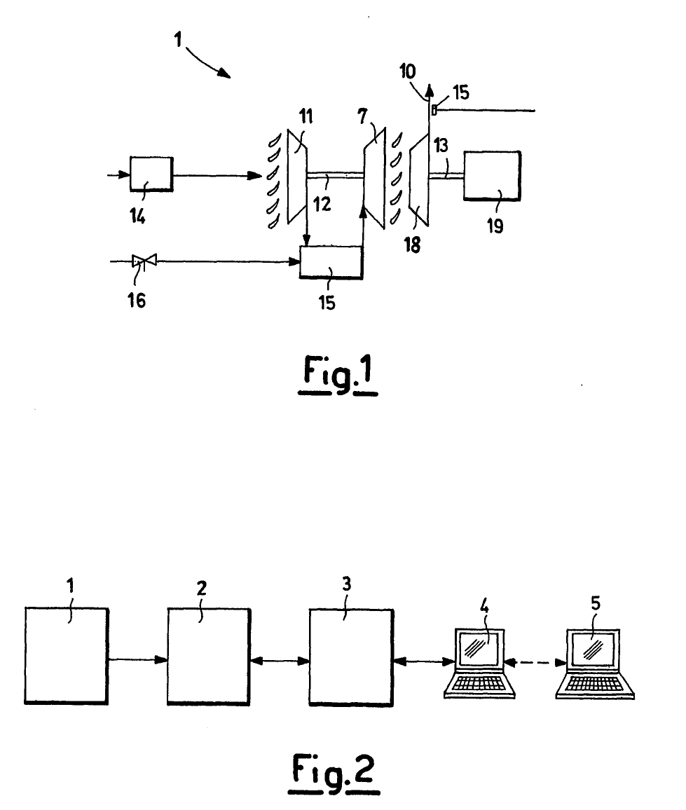

FIG. 1 shows a schematic view of a gas turbine to which the monitoring system according to the present invention can be applied;

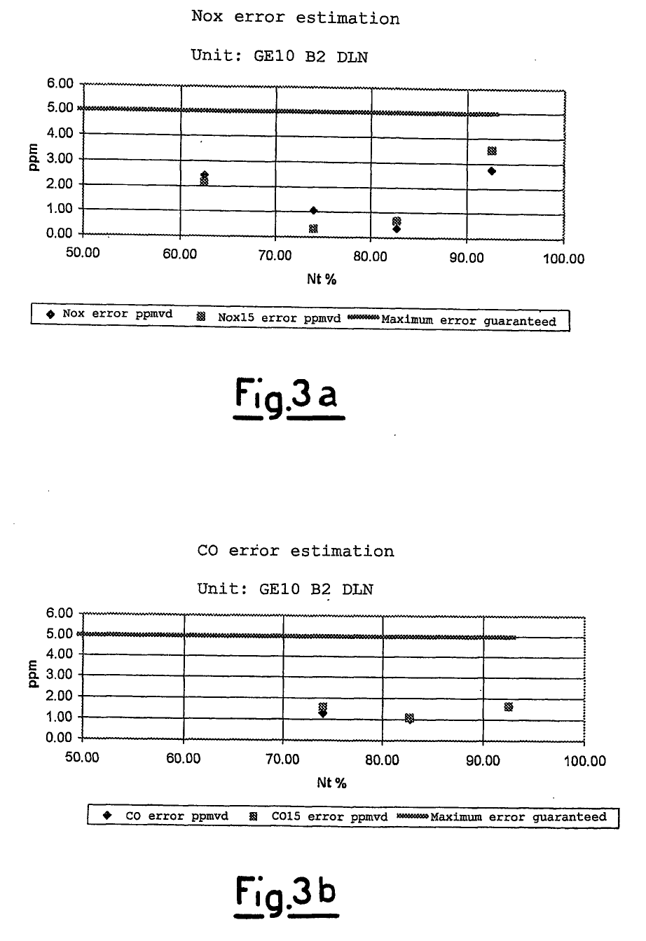

FIG. 2 is a block diagram of the system according to the present invention for estimation and control of the concentrations of pollutant gases at the discharge of a gas turbine; and

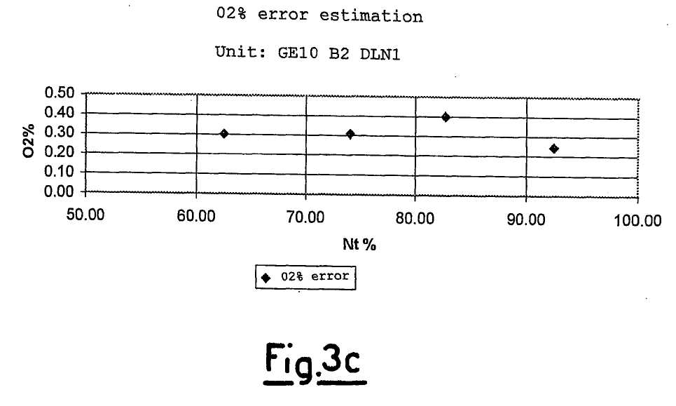

FIGS. 3a-c are graphs which represent the emissions of the said turbine using the system according to the present invention, and the emissions detected by means of an in-line analyser of the emissions themselves.

With reference to the aforementioned figures, a gas turbine 1 with a double shaft comprises a compressor 11 which is rotated by a first shaft 12, which compresses the air sucked in via a filter 14 and conveys it to a combustion chamber 15, into which the fuel is inserted by means of the regulating valve 16. The combustion gases expand in the direction of a turbine 7 which is keyed onto the said first shaft 12, such as to supply the rotary movement of the said compressor. Subsequently, the combustion gases expand in a power turbine 18 and are discharged into the atmosphere via the discharge 10. A sensor 15 to measure the temperature of the emissions is also shown.

The operation of the turbine is controlled by a control panel 2, which receives the values measured on the turbine, for example the speed of rotation of the shafts, the temperature of the emissions, and the temperature of the environment in which the turbine is operating, etc.

According to the present invention, by carrying out monitoring, an estimation, and predictive calculation on the basis of these values measured, it is possible to evaluate the emissions of the turbine at the discharge 10 (oxygen, nitric oxide, carbon monoxide).

The system according to the present invention for estimation and control of the concentrations of pollutant gases at the discharge of a gas turbine comprises a unit 3 for acquisition of data from the control panel 2, comprising a data base to store the data, and a local processing unit 4 which processes the said data in association with the said acquisition unit and makes the data available for consultation, for example by means of a corresponding display.

In addition, the system can also comprise a remote processing unit 5 which is connected by means of a conventional communication line, for example a LAN line or an Internet connection, from which the data processed can be consulted, without needing to be in a location in the vicinity of the gas turbine. For example this remote unit can consult the data via the network by means of a common Internet consultation programme (Internet browser).

In its interior the said acquisition unit comprises a calculator, which carries out the processing operations on the said values measured and stores them in a historic data base which takes into account all the processing operations carried out.

The system operates as follows.

The values measured by the control panel can be for example the temperature at the discharge from the compressor, the temperature at the discharge from the power turbine, the pressure of delivery to the compressor, the ambient temperature, the relative humidity of the environment, the molecular weight of the combustible gas, the compressibility of the combustible gas, the delivery by mass of the fuel, the delivery of the combustion air, the speed of rotation of the turbine, etc.

This data relating to the operating state of the turbine, measured by the control panel, can be processed by this calculator, in addition to some parameters and constants used in order to carry out refinement of the measurements, by interfacing the parameters themselves with the data measured.

The said calculator can also advantageously carry out a statistical calculation of the data stored in the historic data base for the operation of the turbine, for example by calculating the averages or calculating quadratic divergences from the averages.

The applicant has carried out tests analysing the aforementioned data, which show that the values calculated for the emissions are substantially similar to the values measured by means of an in-line analyser. In particular, the graphs in FIGS. 3a-c show respectively the emissions of nitric oxides (FIG. 3a), oxygen (FIG. 3b) and carbon monoxide (FIG. 3c).

1. Method for estimation and control of the concentrations of pollutant gases at the discharge of a gas turbine, comprising the following steps:
receipt of a plurality of signals corresponding to data relating to the operating state of the turbine;
processing of this data; and
evaluation of the emissions into the atmosphere from this turbine on the basis of the said data processed.

2. Method according to claim 1, additionally comprising the step of storage of the data processed in order to create a historic file of the emissions from the turbine.

3. Method according to claim 1, wherein the said step of processing this data comprises the step of interfacing this data with refinement parameters.

4. System for estimation and control of the concentrations of pollutant gases at the discharge of a gas turbine, characterised in that it comprises:
an acquisition unit (3) for the data relating to the operating state of the turbine, the said data being detected by a control panel (2) of the turbine; and
a local processing unit (4) which processes the said data in association with the said acquisition unit and makes the data available for consultation, in order to evaluate the emissions by the said turbine into the atmosphere, on the basis of the said data processed.

5. System according to claim 4, wherein the said local processing unit comprises a data base for storage of the data processed.

6. System according to claim 4, also comprising a remote processing unit (5) which is connected to the said local processing unit by means of a telecommunications line.

7. System according to claim 6, wherein the said telecommunications line is an Internet line.

8. System according to claim 7, wherein the said remote unit consults this data by means of an Internet consultation programme.

9. System according to claim 4, wherein the said local processing unit comprises a calculator which carries out the processing of this data.

10. System according to claim 9, wherein the said calculator carries out statistical calculation of the data stored in the historic data base for operation of the turbine.
